(19) United States
(12) Patent Application Publication
(10) Pub. No.: US 2024/0145062 A1
(43) Pub. Date: May 2, 2024

(54) SYSTEM AND METHOD FOR POSTURE CORRECTION EXERCISE RECOMMENDATION OF ARTIFICIAL INTELLIGENCE BY USING WEARABLE DEVICE

(71) Applicant: PostureAI Inc., Incheon (KR)

(72) Inventor: Eun Young LEE, Seongnam-si Gyeonggi-do (KR)

(73) Assignee: PostureAI Inc., Incheon (KR)

(21) Appl. No.: 18/534,802

(22) Filed: Dec. 11, 2023

(30) Foreign Application Priority Data
Nov. 1, 2022 (KR) 10-2022-0144007

(51) Int. Cl. G16H 20/30; A61B 5/00; G06N 3/006; G06N 3/04; G16H 40/67

(52) U.S. Cl. CPC G16H 20/30; A61B 5/4561; A61B 5/6823; A61B 5/6831; G06N 3/006; G06N 3/04; G16H 40/67; A61B 5/742; A61B 5/7455

(57) ABSTRACT

Provided are a system and method for artificial intelligence (AI) posture correction exercise recommendation using a wearable device. The system includes wearable devices which are worn and used by users for posture measurement and include sensors for sensing the users' postures in 360° directions and a posture correction exercise recommendation device which generates big data by collecting 360°-direction sensing data provided by the wearable devices, analyzes a direction in which a body of each of the users tilts in the 360° directions by analyzing the generated big data through an AI program, and recommends a customized posture correction exercise to a user terminal of each of the users on the basis of analysis results.

### CROSS-REFERENCE TO RELATED APPLICATION

This application claims priority to Korean Patent Application No. 10-2022-0144007 filed on Nov. 1, 2022, the entire contents of which are herein incorporated by reference.

### BACKGROUND

### 1. Field of the Invention

The present invention relates to a system and method for artificial intelligence (AI) posture correction exercise recommendation using a wearable device, and more particularly, to a system and method for AI posture correction exercise recommendation using a wearable device, in which a user's posture data is collected through a wearable device, such as a posture corrector shirt, a posture measurement sensor, or the like, the posture data is analyzed using a deep learning program or the like of AI, and a customized exercise is prescribed according to AI data analysis.

### 2. Discussion of Related Art

Among the common diseases of modern people, diseases related to the musculoskeletal system are frequently caused by a lifestyle habit of maintaining poor posture or the same posture for a long period of time due to the increase in sedentary lifestyles, computer-based work environments, and increased use of portable devices such as smartphones, tablets, and the like. In particular, modern people who mostly have a sedentary lifestyle often repeat the posture of lowering their head or bending their back forward. When such a posture continues, the head gradually turns forward from the center of the body, the shoulders slump, and the back bends. Also, muscles connected to the neck muscles become stiff due to tension in the neck muscles caused by the weight of the head, and muscles in the shoulders are constantly tense to support the weight of the head, which easily leads to fatigue. In addition, when the back is bent, the joints of the spine turn backward from the center of the body, and it is easy for the center of the body to shift or tilt left or right.

Since maintaining correct posture is very important to modern people, chairs, desks, and the like are being made ergonomically for a sedentary lifestyle, and technologies for posture correction are emerging these days.

However, users still cannot receive appropriate feedback for posture correction, and posture correction suitable for each individual user is not properly made.

### RELATED ART DOCUMENTS

### Patent Documents

(Patent Document 1) Korean Patent Publication No. 10-1480026 (Dec. 31, 2014)
(Patent Document 2) Korean Patent Publication No. 10-2310852 (Oct. 1, 2021)
(Patent Document 3) Korean Patent Application Publication No. 10-2022-0085445 (Jun. 22, 2022)

### SUMMARY OF THE INVENTION

The present invention is directed to collecting a user's posture data through a wearable device, such as a posture corrector shirt, a posture measurement sensor, or the like, analyzing the posture data using, for example, an artificial intelligence (AI) deep learning program or the like, and prescribing a customized exercise according to AI data analysis.

Objects of the present invention are not limited to that described above, and other objects which have not been described can be clearly understood by those of ordinary skill in the art from the following description.

According to an aspect of the present invention, there is provided a system for AI posture correction exercise recommendation using a wearable device, the system including wearable devices which are worn and used (onboarding methods) by users for posture measurement and include sensors for sensing the users' postures in 360° directions and a posture correction exercise recommendation device which generates big data by collecting 360°-direction sensing data provided by the wearable devices, analyzes a direction in which a body of each of the users tilts in the 360° directions by analyzing the generated big data through an AI program, and recommends a customized posture correction exercise to a user terminal of each of the users on the basis of analysis results.

The wearable devices may include a belt-shaped posture measurement device that is worn on the center of a user's chest to measure posture forward or backward, left or right, and in 360° directions.

The posture correction exercise recommendation device may generate and provide images that visualize directions in which the users' bodies tilt in the 360° directions according to service requests from the user terminals.

The posture correction exercise recommendation device may visualize and provide daily activity data, which is generated by separating a time of maintaining correct posture from a total usage time of the wearable device for posture measurement, as a graph according to a service request from the user terminal.

When recommending the customized posture correction exercise to the user terminal of each of the users, the posture correction exercise recommendation device may provide a video related to the posture correction exercise or transmit uniform resource locator (URL) information for accessing the posture correction exercise to the user terminal.

The posture correction exercise recommendation device may provide a notification to the user terminal of the user when the user wearing the wearable device deviates from a preset standard posture, and may score and notify of each user's daily posture when there is a request from the user terminal.

According to another aspect of the present invention, there is provided a method of AI posture correction exercise recommendation using a wearable device, the method including providing, by wearable devices which are worn and used by users for posture measurement and include sensors for sensing the users' postures in 360° directions, sensing data obtained by sensing the users' postures in the 360° directions and generating, by a posture correction exercise recommendation device, big data by collecting the 360°-direction sensing data provided by the wearable devices, analyzing a direction in which a body of each of the users tilts in the 360° directions by analyzing the generated big data through an AI program, and recommending a customized posture correction exercise to a user terminal of each of the users on the basis of analysis results.

The recommending of the customized posture correction exercise may include collecting sensing data obtained by measuring posture forward or backward, left or right, and in 360° directions from a belt-shaped posture measurement device that is worn on the center of a user's chest.

The recommending of the customized posture correction exercise may include generating and providing images that visualize directions in which the users' bodies tilt in the 360° directions according to service requests from the user terminals.

The recommending of the customized posture correction exercise may include visualizing and providing daily activity data, which is generated by separating a time of maintaining correct posture from a total usage time of the wearable device for posture measurement, as a graph according to a service request from the user terminal.

The recommending of the customized posture correction exercise may include, when the customized posture correction exercise is recommended to the user terminal of each of the users, providing a video related to the posture correction exercise or transmitting URL information for accessing the posture correction exercise to the user terminal.

The recommending of the customized posture correction exercise may include providing a notification to the user terminal of the user when the user wearing the wearable device deviates from a preset standard posture, and scoring and notifying of each user's daily posture when there is a request from the user terminal.

Other details of exemplary embodiments are included in detailed description and drawings.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is a diagram illustrating a system for artificial intelligence (AI) posture correction exercise recommendation using a wearable device according to an exemplary embodiment of the present invention;

FIGS. 2A, 2B, 2C, 2D are exemplary diagrams showing various types of wearable devices of FIG. 1;

FIGS. 3A, 3B, 3C are exemplary diagrams showing posture AI conversation and user interface (UI) screens used in a user terminal of FIG. 1;

FIG. 4 is an exemplary block diagram illustrating a detailed structure of a posture correction exercise recommendation device of FIG. 1; and

FIG. 5 is a flowchart illustrating a method of AI posture correction exercise recommendation using a wearable device according to an exemplary embodiment of the present invention.

### DETAILED DESCRIPTION OF EXEMPLARY EMBODIMENTS

Hereinafter, various exemplary embodiments of the present invention will be described in detail with reference to the accompanying drawings. Advantages and features of the present invention and methods of achieving the same will become apparent with reference to the exemplary embodiments that are described in detail in conjunction with the accompanying drawings. However, the present invention is not limited to the exemplary embodiments set forth herein and can be implemented in various different forms. The exemplary embodiments are provided only to make the disclosure of the present invention complete and fully convey the scope of the present invention to those with ordinary knowledge in the technical field of the present invention. The present invention is defined only by the category of the claims.

Shapes, sizes, ratios, angles, numbers, and the like shown in the drawings for describing the exemplary embodiments of the present invention are illustrative, and the present invention is not limited thereto. Throughout the specification, the same reference numeral refers to the same component. When it is determined that a detailed description of associated known technology unnecessarily obscures the subject matter of the present invention, the detailed description will be omitted. As used herein, the terms “comprise,” “include,” “have,” and the like do not preclude the presence or addition of other parts unless used along with the term “only.” The singular forms include the plural forms as well unless the context clearly indicates otherwise.

Components are interpreted as including an error range even when there is no explicit description thereof.

When a positional relationship is described, for example, when a positional relationship between two part is described using “on,” “over,” “under,” “beside” or the like, one or more other parts may be interposed between the two parts unless the term “immediately” or “directly” is used.

When an element or layer is referred to as being “on” another element or layer, still another element or layer may be interposed therebetween.

Although terms including “first,” “second,” and the like may be used for describing various components, the components are not limited by the terms. These terms are merely used for distinguishing one component from another. Therefore, in the following description, a first component may be a second component within the technical spirit of the present invention.

Throughout the specification, the same reference numeral refers to the same component.

The size and thickness of each element shown in the drawings are depicted for the convenience of description, and the present invention is not necessarily limited thereto.

Features of various embodiments of the present invention may be partially or overall coupled to or combined with each other and may be variously operated in conjunction with each other and driven technically as those skilled in the art can sufficiently understand. Embodiments may be implemented independently from each other or implemented together in a dependent relationship.

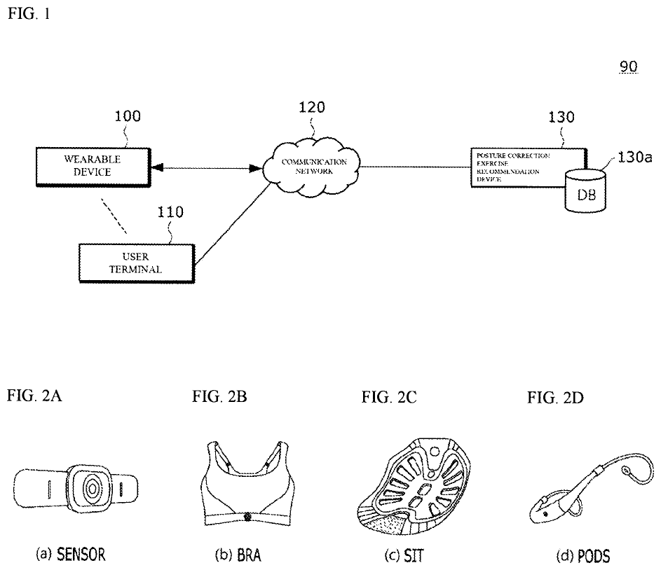

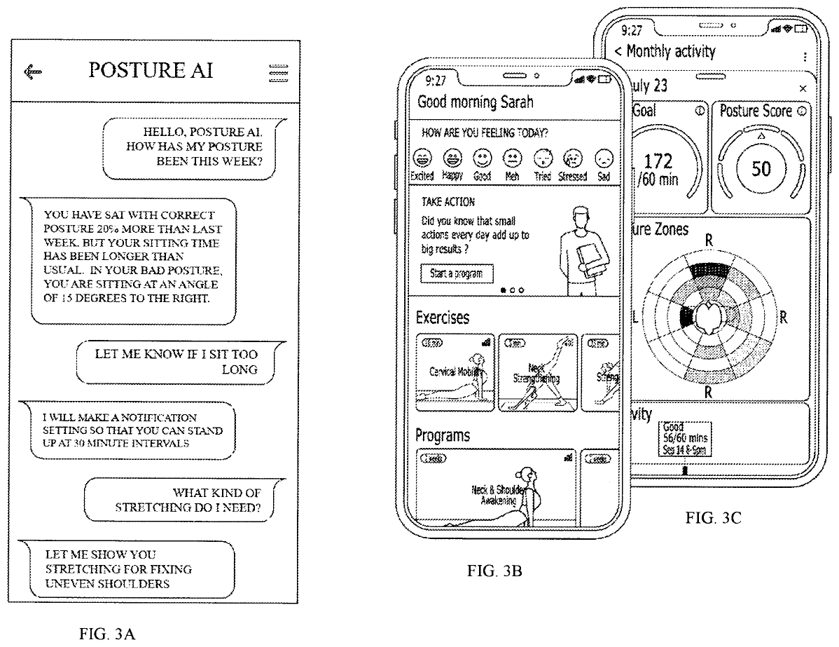

FIG. 1 is a diagram illustrating a system for artificial intelligence (AI) posture correction exercise recommendation using a wearable device according to an exemplary embodiment of the present invention, FIGS. 2A, 2B, 2C, 2D are exemplary diagrams showing various types of wearable devices of FIG. 1, and FIGS. 3A, 3B, 3C are exemplary diagrams showing posture AI conversation and user interface (UI) screens used in a user terminal of FIG. 1.

As shown in FIG. 1, a system 90 for AI posture correction exercise recommendation using a wearable device (hereinafter, “posture correction exercise recommendation system”) according to the exemplary embodiment of the present invention includes all or some of a wearable device 100, a user terminal 110, a communication network 120, and a posture correction exercise recommendation device 130.

Here, “includes all or some” means that the posture correction exercise recommendation system 90 does not include some components, such as the user terminal 110, or all or some components of the posture correction exercise recommendation device 130 are integrated with the wearable device 100 or a network device (e.g., a wireless switching device or the like) constituting the communication network 120. To help with comprehensive understanding of the present invention, the posture correction exercise recommendation system 90 will be described as including all the components.

As shown in FIG. 2, the wearable device 100 may be any of various types of devices for measuring a user's posture according to an exemplary embodiment of the present invention. In detail, to collect the user's daily data about sitting, standing, and moving, the wearable device 100 may be a sensor device worn on a user's waist or the center of the chest (e.g., the pit of the user's stomach) which is the center of the body, a clothing-type device, such as a sports bra or the like, worn on the torso, a smart seat which is put or installed on a chair where the user sits, a turtle neck syndrome fixer, or the like as shown in FIGS. 2A to 2D. The smart seat is related to sitting, and the sports bra and the turtle neck syndrome fixer may be related to moving. Further, the wearable device 100 according to the exemplary embodiment of the present invention may be a hybrid product in which a lifestyle wearable device is combined with a musculoskeletal medical device, and thus embodiments of the present invention are not limited to any one type of wearable device.

The wearable device 100 may include a wearable sensor for measuring the user's posture. For example, in the case of a smart seat, pressure sensors may be included to determine in which part of a chair a higher pressure is measured when a user sits on the chair. When a higher pressure is measured at a pressure sensor on the front side, the user's posture may be considered as tilting forward. Above all, a sensor installed on the wearable device 100 according to the exemplary embodiment of the present invention preferably includes a sensor for sensing movement or posture of the user's body in 360° directions. A 3-axis gyro sensor for measuring the 360° directions may be representatively used. In detail, a 3-axis acceleration sensor or the 3-axis gyro sensor may be used, which may be an inertial measurement unit (IMU). With this, it is possible to measure a user's posture angle, posture angular speed, acceleration, and the like.

Accordingly, in an exemplary embodiment of the present invention, it is possible to measure a forward or backward direction and a left or right direction through the 3-axis gyro sensor, the 3-axis acceleration sensor, or the like and measure movement in the 360° directions, that is, the body posture. Since the wearable device 100 may employ various types of sensors for measuring a user's posture region forward, backward, left, and right, and in the 360° directions, embodiments of the present invention are not limited to any one type of sensor. According to an exemplary embodiment of the present invention, sensing data acquired through the sensor of the wearable device 100 is not limited to posture information about a forward or backward tilt, and only when posture information about the left, right, and 360° directions is acquired, it is possible to recommend an appropriate posture correction exercise. Accordingly, posture data related to the 360° directions may be necessarily required for an exercise recommendation service according to an exemplary embodiment of the present invention.

The user terminal 110 may be not only a smartphone shown in FIGS. 3A, 3B, 3C but also any of various types of devices. For example, the user terminal 110 may be another wearable device worn on the user's wrist or the like or may be a mobile device, such as a tablet personal computer (PC), or even a PC-based terminal such as a desktop computer or laptop computer. Therefore, according to exemplary embodiments of the present invention, the user terminal 110 is not limited to a specific type.

The user terminal 110 according to the exemplary embodiment of the present invention may access the posture correction exercise recommendation device 130 through the communication network 120 of FIG. 1, and each of the users may check analysis results of data related to their posture. A user experience/user interface (UX/UI) screen provided to the user terminal 110 may vary. According to a representative exemplary embodiment of the present invention, the user terminal 110 may receive an exercise recommendation service based on AI analysis. For example, exercise recommendation may be provided in the form of a video, or uniform resource locator (URL) information for accessing the video may be provided.

Also, the user terminal 110 may receive a bad-posture vibration notification, that is, when a bad posture is detected, a notification signal may be transmitted to vibrate the user terminal 110 or the wearable device 100. Also, individual posture habit analysis results in accordance with analysis results of collected data can be checked in the form of a graph, a diagram, and the like. For example, when a user is watching television (TV), the user's posture may be detected, and a notification service, such as the notification “your shoulders are bent too much and you need to stretch your deltoids!” may be provided through the user terminal 110. The notification may be provided in the form of a text message or a messenger-based chatting service, or the corresponding content may be output in the form of voice through a speaker. When a nearby AI speaker is detected, the voice may be output through the AI speaker. For example, the user terminal 110 and the AI speaker may perform direct communication, such as beacon communication. In this way, the posture correction exercise recommendation device 130 can recognize what kind of surrounding device is there, and thus output voice through the AI speaker according to the recognition result.

In addition, the user terminal 110 may check posture data analysis results through an application (hereinafter “app”) for using a service according to an exemplary embodiment of the present invention. As a representative analysis result, a posture score may be provided. Daily posture may be scored, and the score may be checked. A method of calculating the score may vary and is not limited to any one method. For example, assuming that a total time of maintaining correct posture is 10 hours, when the user maintains correct posture for only five hours and is not in correct posture for the remaining period of time, the daily score may be 50. The user terminal 110 may check daily activity data, in which a time of maintaining correct posture is separated from a total time of using the wearable device 100, in a way other than the score. The daily activity data can be checked in the form of a histogram.

Further, the user terminal 110 may check real-time posture data of the user periodically on a daily, weekly, or monthly basis. Above all, analysis results of the users' 360° posture regions may be checked using 360° sensing data acquired from the wearable device 100. A direction in which the body tilts may be visualized at an angle of 0° to 360° and thus can be easily recognized. This is shown well in the lower portion of FIG. 3C. In a UI screen shown in FIG. 3C, a front portion is displayed dark (or in a color), which means that the corresponding user has a posture of tilting the body slightly forward.

Moreover, the user terminal 110 may communicate with a posture AI chatbot or the like. In this way, a user may be coached for AI-based one-to-one customized posture (see FIG. 3A). In addition, the posture correction exercise recommendation device 130 may train an AI program of the AI chatbot with answers to various questions in advance, and the user terminal 110 may receive an answer to a question. Like a human, the AI program recognizes a sentence in the form of text and thus can predict and provide an answer to the sentence. In this regard, the AI program differs from a rule-based chatbot according to the related art. In other words, according to the related art, when there is a question from a user, the chatbot cannot answer the question without rules. However, AI can make a prediction on the basis of analysis results of the question. Accordingly, the AI can continue the conversation by providing a supposed answer and provide a correct answer to the question of the user through data learning. Therefore, in the case of the posture AI chatbot, when there is a question from a user, an appropriate answer to the question is made through an AI deep learning program. Posture AI according to an exemplary embodiment of the present invention may learn data related to posture correction or exercise recommendation in advance and provide appropriate question-related answers to users on the basis of the learned data. However, embodiments of the present invention are not limited to such answers related to posture correction or exercise recommendation. Relevant content is shown in FIG. 3A, and the user terminal 110 can use a posture correction subscription service as shown in FIG. 3B. Through a corresponding subscription service screen, it is possible to check data, such as a video and the like, for AI-based one-to-one customized posture coaching.

The communication network 120 may include both wired and wireless communication networks. For example, as the communication network 120, wired and wireless Internet networks may be used or interoperate. Here, the wired network is an Internet network, such as a cable network or a public switched telephone network (PSTN), and the wireless network is a code division multiple access (CDMA) network, a wideband CDMA (WCDMA) network, a Global System for Mobile Communications (GSM) network, an Evolved Packet Core (EPC) network, a Long-Term Evolution (LTE) network, a wireless broadband (WiBro) network, or the like. The communication network 120 according to the exemplary embodiment of the present invention is not limited thereto, and an access network of a next-generation mobile communication system, for example, a cloud computing network under a cloud computing environment, a Fifth Generation (5G) network, or the like, may be used. When the communication network 120 is a wired communication network, an access point in the communication network may access a telephone exchange or the like. On the other hand, when the communication network 120 is a wireless communication network, an access point in the communication network may process data by accessing a serving general packet radio service (GPRS) support node (SGSN) or a gateway GPRS support node (GGSN) run by a communication service company or may process data by accessing various relay devices, such as a base transceiver station (BTS), a node base station (NodeB), an evolved nodeB (e-NodeB), and the like.

The communication network 120 may include an access point. The access point is a small base station, such as a femto or pico base station, frequently installed in a building. According to classifications of small base stations, a femto or pico base station is determined depending on how many wearable devices 100, user terminals 110, and the like are connected thereto at the maximum. The access point may include a short-range communication module for performing short-range communication, such as ZigBee, Wi-Fi, and the like, with the wearable device 100, the user terminal 110, or the like. The access point may use transmission control protocol/Internet protocol (TCP/IP) or the real-time streaming protocol (RTSP). Here, the short-range communication may be performed according to various standards of radio frequency (RF) communication, ultra-wideband (UWB) communication, and the like such as Wi-Fi, Bluetooth, ZigBee, Infrared Data Association (IrDA), ultra-high frequency (UHF), and very high frequency (VHF). Accordingly, the access point may extract the location of a data packet, designate the best communication path to the extracted location, and transmit the data packet to the next device, for example, the posture correction exercise recommendation device 130. The access point may share several lines in a general network environment and includes, for example, a router, a repeater, a relay device, and the like.

The posture correction exercise recommendation device 130 may collect each user's daily data about sitting, standing, and moving through the wearable device 100 of FIG. 1, generate big data by storing the collected data in a database (DB) 130a of FIG. 1, analyze the big data through the AI deep learning program or the like, and then provide a service of posture correction, exercise recommendation, or the like to the users. To this end, the posture correction exercise recommendation device 130 may construct an all-in-one posture integration solution so that various services (e.g., exercise recommendation, posture correction subscription, and the like) may be provided through one location. For example, the posture correction exercise recommendation device 130 may measure the user's posture in real time and directly provide a vibration notification to the wearable device 100 or the user terminal 110 carried by the user when bad posture is detected. Also, when the user carrying the user terminal 110 requests analysis results related to his or her posture habit, the posture correction exercise recommendation device 130 may provide the analysis results on the basis of data collection and analysis. In addition, the posture correction exercise recommendation device 130 can provide the posture correction subscription service, perform AI-based one-to-one customized posture coaching through the posture AI chatbot and messenger-based chatting, and recommend an exercise for posture correction to the user terminal 110.

Since the posture correction exercise recommendation device 130 according to the exemplary embodiment of the present invention may collect sensing data related to forward or backward, leftward or rightward, and 360° posture or movement from the wearable device 100, it is possible to provide an exercise treatment service on the basis of the sensing data. In this way, the posture correction exercise recommendation device 130 may track a posture habit, periodically determine a change in the posture habit, perform a one-to-one coaching operation when posture correction is necessary, and prescribe a customized rehabilitation exercise when it is necessary to correct the posture habit. The posture correction exercise recommendation device 130 may provide an integrated posture correction solution that is a solution related to such an operation. For example, the posture correction exercise recommendation device 130 can estimate a user's posture habit through big data analysis and perform a prevention operation by notifying the user of the possibility of a major problem and performing a coaching operation for posture correction before the major problem develops. In this process, for the purpose of coaching, the posture correction exercise recommendation device 130 may recommend a specific exercise to the user and receive data about whether the user does the recommended exercise. It is possible to check whether the user does the posture correction exercise according to the coaching by analyzing sensing data acquired from the wearable device 100 or the like. In this way, coaching may be performed in various ways according to users' situations.

As described above, the posture correction exercise recommendation device 130 may perform correct posture maintenance time analysis, periodic data analysis, posture score provision, and 360° posture region analysis operations. In the correct posture maintenance time analysis operation, the posture correction exercise recommendation device 130 may separate, that is, determine, a correct posture maintenance time from a total time of using the wearable device 100, generate daily activity data, and provide analysis results to the user terminal 110. Also, the posture correction exercise recommendation device 130 may score daily posture by analyzing posture data and provide the score to the user terminal 110. Reference data which is criteria for posture may be constructed in the DB 130a in advance. Accordingly, the posture correction exercise recommendation device 130 may perform an operation of calculating the user's posture correction range or posture correction index by comparing the posture data with the reference data and scoring the posture data on the basis of the posture correction range or index or other operations. Such standard posture may be determined by analyzing images of reference people, for example, experts, and comparing the analysis results. Further, the posture correction exercise recommendation device 130 acquires sensing data about 360° body tilts and the like and thus may analyze the sensing data, visualize a direction in which the body tilts at an angle of 0° to 360° and provide a visualization screen to the user terminal 110 of the user. This has already been described with reference to FIG. 3C.

Meanwhile, the posture correction exercise recommendation device 130 according to an exemplary embodiment of the present invention may recognize the user's current state according to the type of wearable device 100 and recommend an appropriate exercise for the current state. For example, when the turtle neck syndrome fixer shown in FIG. 2D is used, a video related to an appropriate neck exercise may be recommended, and when the smart seat shown in FIG. 2C is used, a video of an appropriate exercise that may be performed in a sitting position may be recommended. To provide such a service, the posture correction exercise recommendation device 130 may create recommendation videos, such as appropriate videos that may be provided according to each type of wearable device 100 and the user's posture, and provide the service on the basis of the recommendation videos. In other words, the posture correction exercise recommendation device 130 may determine the user's current state and the type of wearable device 100 from currently received sensing data. Assuming that tens of recommendation videos may be recommended according to various postures, when it is determined that the user is in a sitting state, a recommendation video that matches a posture analysis result of the user in the sitting state may be extracted and provided to the user terminal 110 of the user.

Moreover, according to an exemplary embodiment of the present invention, it is possible to provide a posture correction service through the posture correction exercise recommendation device 130 in various fields related to posture correction, such as golf swing posture correction, fitness and virtual training, weightlifting posture correction, office worker posture correction, musculoskeletal and physical therapy, and the like. Accordingly, embodiments of the present invention are not limited to posture correction operations in daily life.

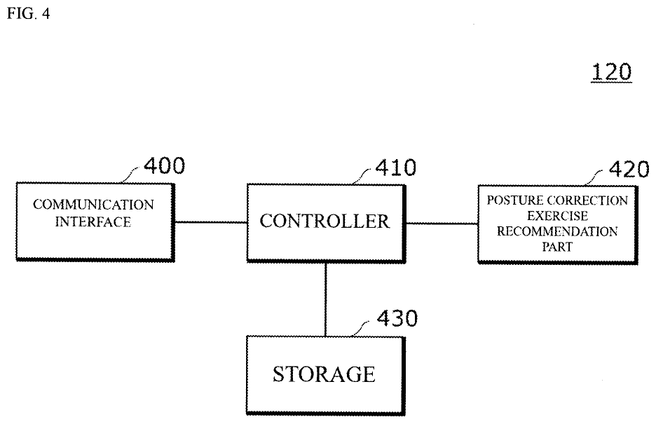

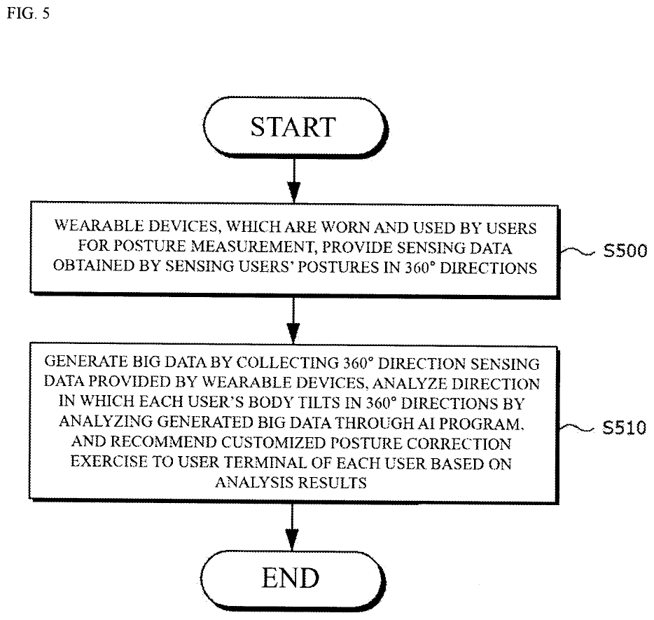

FIG. 4 is an exemplary block diagram illustrating a detailed structure of a posture correction exercise recommendation device of FIG. 1.

As shown in FIG. 4, the posture correction exercise recommendation device 130 according to the exemplary embodiment of the present invention includes all or some of a communication interface 400, a controller 410, a posture correction exercise recommendation part 420, and a storage 430.

Here, “includes all or some” means that the posture correction exercise recommendation device 130 does not include some components, such as the storage 430, or some components of the posture correction exercise recommendation device 130, such as the posture correction exercise recommendation part 420, are integrated with the other components, such as the controller 410. To help with comprehensive understanding of the present invention, the posture correction exercise recommendation device 130 will be described as including all the components.

The communication interface 400 communicates with each of the wearable device 100 and the user terminal 110 of FIG. 1. In a communication process, the communication interface 400 may perform modulation/demodulation operations, encoding/decoding operations, multiplexing/demultiplexing (MUXing/DEMUXing) operations, a resolution conversion operation, and the like. When the user terminals 110 are smartphones, screens of all the user terminals 110 vary in size. Accordingly, the resolution of the screen of FIG. 3B or 3C may be converted so that an optimal screen is provided.

When the wearable device 100 operates and is registered in advance, the communication interface 400 receives and transmits sensing data to the controller 410 in real time. For example, when the user uses the posture measurement device shown in FIG. 2A and the smart seat shown in FIG. 2C to find a movement direction of the body at an angle of 0° to 360°, data collected from the two wearable devices 100 is classified by the wearable devices 100 carried by the user and provided to the controller 410. In other words, when the user registers the wearable device 100 that he or she carries according to an exemplary embodiment of the present invention, device identification information of the wearable device 100 is acquired and stored in the DB 130a of FIG. 1 or the like, and thus it is possible to know which user carries which wearable device 100 from the information. This is because, when operating and transmitting sensing data, the wearable device 100 transmits the device identification information together. In addition, the wearable device 100 may also transmit information on a time at which the sensing data is generated.

Further, when an app is executed in the user terminal 110 of FIG. 1, the communication interface 400 may communicate with the user terminal 110 and provide analysis results of data related to the user's posture according to the user's service request. A UI screen may be representatively provided regarding posture forward or backward, left or right, and in 360° directions. The communication interface 400 may provide a notification for posture correction according to a request from the controller 410 when the posture of the user of the wearable device 100 deviates from the standard posture. When an app is used, a push notification may be provided through the app.

The controller 410 is in charge of overall control operations for the communication interface 400, the posture correction exercise recommendation part 420, and the storage 430 of FIG. 4 constituting the posture correction exercise recommendation device 130 of FIG. 1. When sensing data obtained by measuring each user's posture is representatively received from the communication interface 400, the controller 410 may temporarily store the sensing data in the storage 430 and then provide the sensing data to the posture correction exercise recommendation part 420. In this process, when the posture correction exercise recommendation part 420 processes data in a designated format and provides the processed data to classify and store the data in the DB 130a of FIG. 1, the controller 410 may control the communication interface 400 to systematically classify and store the data in the designated format in the DB 130a.

Also, the controller 410 may perform an operation for bringing specific users' posture data from the DB 130a of FIG. 1 and analyzing the posture data. The analysis operation is performed by the posture correction exercise recommendation part 420. However, the controller 410 may store, in an internal memory thereof, the same analysis program as installed in the posture correction exercise recommendation part 420 and execute the analysis program, and thus an entity that performs the analysis operation is not limited to the posture correction exercise recommendation part 420. Here, the analysis data may be classified and stored in the DB 130a of FIG. 1 under the control of the controller 410. In addition, when the user terminal 110 of FIG. 1 accesses the service and requests various forms of data related thereto, the controller 410 may control the posture correction exercise recommendation part 420 so that a UI screen obtained by graphically processing data of the user is provided to the user terminal 110. A screen related to posture measurement at a precise inclination forward or backward, left or right, and in the 360° directions, a screen for one-to-one coaching based on posture habit tracking, or a video for exercise recommendations based on analysis results of the user's posture measurement data may be representatively provided. For such an operation, the controller 410 may operate in conjunction with the posture correction exercise recommendation part 420.

The posture correction exercise recommendation part 420 may perform various operations according to an exemplary embodiment of the present invention. The posture correction exercise recommendation part 420 may representatively generate analysis results by analyzing sensing data of user-specific posture measurement which is acquired from the wearable device 100. In this process, the posture correction exercise recommendation part 420 may have an AI program installed therein, learn posture-related data in advance, and then perform an operation of measuring a user's posture on the basis of the learning results. In general, learning includes supervised learning and non-supervised learning. To construct training data for supervised learning, a program designer sets data as a standard posture. On the other hand, non-supervised learning is a process in which an AI program continues determining a standard posture on its own by analyzing big data. Accordingly, non-supervised learning requires a large amount of data. Non-supervised learning may also require data related to a standard posture at the initial stage. According to exemplary embodiments of the present invention, supervised learning and non-supervised learning can be used together, and thus the present invention is not limited to any one type of learning. The AI deep learning program may increase accuracy in posture measurement. Therefore, it is possible to recommend posture correction exercises that match accurate results of posture measurement to the user terminals 110 of the users.

Above all, when the posture correction exercise recommendation part 420 generates big data by collecting daily data about sitting, standing, and moving from the wearable devices 100 and analyzes the big data, the users' posture may be precisely measured in the 360° directions. Such 360°-direction posture measurement data may be acquired through the 3-axis gyro sensor or the like of the wearable device 100 and analyzed to recommend a customized exercise in accordance with each user's posture habit.

Since the types of wearable devices 100 carried by the users may vary, the posture correction exercise recommendation part 420 preferably recommends an exercise on the basis of data acquired from the wearable device 100 carried by each of the users. However, the posture correction exercise recommendation part 420 collects and analyzes big data, and thus the accuracy of a posture correction exercise recommendation can be further increased by mutually reflecting data of wearable devices 100 that user A or user B does not carry. In other words, a posture correction exercise recommended on the basis of analysis results of data acquired from 100 people may show higher accuracy than a posture correction exercise recommended on the basis of analysis results of data acquired from five people. Therefore, the posture correction exercise recommendation part 420 can recommend an exercise by comprehensively considering posture measurement data of other users rather than only sensing data of the wearable device 100 carried by the user.

For example, a posture correction exercise may be recommended on the basis of analysis results of the four wearable devices 100 shown in FIGS. 2A, 2B, 2C, 2D according to an exemplary embodiment of the present invention. When a user only carries the posture measurement device shown in FIG. 2A, a posture correction exercise is recommended by reflecting data of the other three wearable devices 110 not carried by the user as well as sensing data of the posture measurement device, in detail, data extracted from many users in common. In this process, the posture correction exercise recommendation part 420 may bring data of users who have the same sex as the user and identical or similar ages, physical conditions, job types, and the like and reflect the data in the exercise recommendation. Accordingly, exercise A is recommended on the basis of one wearable device 100, whereas exercise B may be recommended on the basis of data of other users as well as data of the wearable device 100. In other words, exercise A is a result that only reflects sensing data related to the neck, and exercise B is a recommended result that comprehensively reflects relationships between the neck, the spine, the waist, and the like.

The storage 430 may temporarily store various types of data processed under the control of the controller 410. When posture measurement data of the wearable device 100 is temporarily stored, the storage 430 may provide the posture measurement data to the posture correction exercise recommendation part 420 to perform data analysis.

In addition to the above description, the communication interface 400, the controller 410, the posture correction exercise recommendation part 420, and the storage 430 of FIG. 4 may perform various operations. Other details have been sufficiently described above and thus will not be reiterated.

The communication interface 400, the controller 410, the posture correction exercise recommendation part 420, and the storage 430 of FIG. 4 according to the exemplary embodiment of the present invention may be configured as hardware modules physically separated from each other, but each module may store software for performing the operations therein and execute the software. However, the software is a set of software modules, and each module can be hardware. Accordingly, the configuration is not limited to software or hardware. For example, the storage 430 may be a storage or memory which is hardware. However, it is also possible to reposit information in a software manner, and thus the storage 430 is not limited to hardware.

Meanwhile, according to another exemplary embodiment, the controller 410 may include a central processing unit (CPU) and a memory and may be formed into one chip. The CPU may include a control circuit, an arithmetic logic unit (ALU), an instruction interpreter, a registry, and the like, and the memory may include a random access memory (RAM). The control circuit may perform a control operation, the ALU may perform a calculation operation on binary bit information, and the instruction interpreter may include an interpreter, a compiler, or the like and perform an operation of converting a high-level language into the machine language or converting the machine language into a high-level language, and the registry may take part in data storage in a software manner. According to the above configuration, in the initial stage of an operation of the posture correction exercise recommendation device 130, a program stored in the posture correction exercise recommendation part 420 may be copied and loaded onto the memory, that is, the RAM, and then executed so that a data calculation processing rate can be rapidly increased. In the case of a deep learning model, the program may be loaded onto a graphics processing unit (GPU) memory rather than the RAM and executed with a processing rate which is increased using the GPU.

FIG. 5 is a flowchart illustrating a method of AI posture correction exercise recommendation using a wearable device according to an exemplary embodiment of the present invention.

Referring to FIGS. 1 and 5 together for convenience of description, the wearable device 100 according to the exemplary embodiment of the present invention is a device that is worn or used by a user for posture measurement and includes a sensor for sensing the user's posture in the 360° directions and may provide sensing data obtained by sensing the user's posture in the 360° directions (S500). When the wearable device 100 is a smart seat, a pressure may be measured, and thus it is possible to provide sensing data obtained from a pressure sensor at a specific location. In this case, information on the locations of pressure sensors in the smart seat may be stored in advance, and the posture correction exercise recommendation device 130 may recognize which location corresponds to the sensing data using the stored information.

Also, the posture correction exercise recommendation device 130 generates (or creates) big data by collecting 360°-direction sensing data provided by the wearable device 100, analyzes a direction in which each user's body tilts in the 360° directions by analyzing the generated big data through an AI program, and recommends a customized posture correction exercise to the user terminal 110 of each user on the basis of the analysis results (S510).

Here, the recommendation of a customized posture correction exercise has been sufficiently described above. In other words, in the case of recommending a customized posture correction exercise to each user, when data for exercise recommendation is constructed on the basis of a total of four wearable devices 100, a customized posture correction exercise may be recommended to the corresponding user with reference to not only data from the wearable device 100 carried by the user but also data from other wearable devices 100 that are not carried by the user. In this case, analysis results of data of users who have the same sex as the user or identical or similar ages or data of users who have the same or similar life habits or job types are used as the reference data. In this way, the accuracy of a recommended posture correction exercise can be increased.

In addition to the above description, the wearable device 100, the user terminal 110, and the posture correction exercise recommendation device 130 of FIG. 1 may perform various operations. Other details have been sufficiently described above and thus will not be reiterated.

Meanwhile, even when all components constituting an exemplary embodiment of the present invention are described as being combined into one or operating in combination, the present invention is not necessarily limited thereto. In other words, all the components may also be selectively combined into one or more components and operate without departing from the scope of the present invention. In addition, each component may be implemented as one piece of independent hardware, and some or all of the components may be selectively combined and implemented as a computer program having a program module that performs some or all functions combined in one or more pieces of hardware. Codes and code segments constituting the computer program may be easily inferred by those of ordinary skill in the art to which the present invention pertains. The computer program is stored in non-transitory computer readable media and read and executed by a computer so that an embodiment of the present invention can be implemented.

The non-transitory computer readable media are not media that store data therein for a short period of time, such as a register, a cache, a memory, or the like, but media that semi-permanently store data therein and are readable by a device. In detail, the foregoing program may be stored and provided in a non-transitory computer readable medium such as a compact disc (CD), a digital versatile disc (DVD), a hard disk, a Blu-ray disc, a Universal Serial Bus (USB) memory, a memory card, a read-only memory (ROM), or the like.

According to the present invention, it is possible to collect and analyze users' daily data about sitting, standing, and moving and prescribe customized exercises to the users according to AI data analysis.

Also, according to the present invention, it is possible to provide a posture correction subscription service for AI-based one-to-one customized posture coaching.

Further, according to the present invention, it is possible to carry out self-directed health management even in a non-face-to-face manner in order to prevent spinal diseases and the like.

Effects of the present invention are not limited to those described above, and various other effects are included in the specification.

Although exemplary embodiments of the present invention have been illustrated and described above, the present invention is not limited to the specific embodiments described above. The present invention may be variously modified by those of ordinary skill in the art to which the present invention pertains without departing from the scope of the present invention described in the claims, and such modifications should be construed as falling within the technical spirit of the present invention.

Although the present invention has been described above with reference to specific details, such as specific components and the like, limited embodiments, and drawings, these are only provided to help with overall understanding of the present invention. The present invention is not limited to the embodiments, and those of ordinary skill in the art to which the present invention pertains can make various alterations and modifications from the descriptions.

Therefore, the spirit of the present invention should not be limited to the foregoing embodiments, and the following claims and all modifications which are equivalent thereto should construed as falling within the scope of the present invention.

What is claimed is:

1. A system for artificial intelligence (AI) posture correction exercise recommendation using a wearable device, the system comprising:
wearable devices which are worn and used by users for posture measurement and include sensors for sensing the users' postures in 360° directions; and
a posture correction exercise recommendation device which generates big data by collecting 360°-direction sensing data provided by the wearable devices, analyzes a direction in which a body of each of the users tilts in the 360° directions by analyzing the generated big data through an AI program, and recommends a customized posture correction exercise to a user terminal of each of the users on the basis of analysis results,
wherein the user terminal receives a bad-posture vibration notification, vibrates the wearable device by transmitting a notification signal regarding the bad-posture vibration notification to the wearable device, and displays individual posture habit analysis results as a graph or a diagram in accordance with the analysis results of the collected data,
the user terminal performs direct communication with a nearby AI speaker using a beacon and outputs, when the bad-posture vibration notification is received, a received result to the AI speaker,
the user terminal provides real-time posture data of the user periodically on a daily, weekly, or monthly basis,
the posture correction exercise recommendation device trains an AI program of an AI chatbot with answers to various questions in advance so that the user terminal receives an answer to a question from the posture correction exercise recommendation device,
the posture correction exercise recommendation device provides analysis results related to each individual's posture habit on the basis of data collection and analysis to the user terminals when the users carrying the user terminals request the analysis results, and
the posture correction exercise recommendation device recognizes a current state of the user according to a type of wearable device and recommends an exercise, which is appropriate for the user in the current state, to the user.

2. The system of claim 1, wherein the wearable devices include belt-shaped posture measurement devices that are worn on centers of the users' chests to measure postures forward or backward, left or right, and in 360° directions.

3. The system of claim 1, wherein the posture correction exercise recommendation device generates and provides images that visualize directions in which the users' bodies tilt in the 360° directions according to service requests from the user terminals.

4. The system of claim 1, wherein the posture correction exercise recommendation device visualizes and provides daily activity data, which is generated by separating a time of maintaining correct posture from a total usage time of the wearable device for posture measurement, as a graph according to a service request from the user terminal.

5. The system of claim 1, wherein, when the posture correction exercise recommendation device recommends the customized posture correction exercise to the user terminal of each of the users, the posture correction exercise recommendation device provides a video related to the posture correction exercise or transmits uniform resource locator (URL) information for accessing the posture correction exercise to the user terminal.

6. The system of claim 1, wherein the posture correction exercise recommendation device provides a notification to the user terminal of the user when the user wearing the wearable device deviates from a preset standard posture, and scores and notifies of each user's daily posture when there is a request from the user terminal.

7. A method of artificial intelligence (AI) posture correction exercise recommendation using a wearable device, the method comprising:
providing, by wearable devices which are worn and used by users for posture measurement and include sensors for sensing the users' postures in 360° directions, sensing data obtained by sensing the users' postures in the 360° directions; and
generating, by a posture correction exercise recommendation device, big data by collecting the 360°-direction sensing data provided by the wearable devices, analyzing a direction in which a body of each of the users tilts in the 360° directions by analyzing the generated big data through an AI program, and recommending a customized posture correction exercise to a user terminal of each of the users on the basis of analysis results,
wherein the user terminal receives a bad-posture vibration notification, vibrates the wearable device by transmitting a notification signal regarding the bad-posture vibration notification to the wearable device, and displays individual posture habit analysis results as a graph or a diagram in accordance with the analysis results of the collected data,
the user terminal performs direct communication with a nearby AI speaker using a beacon and outputs, when the bad-posture vibration notification is received, a received result to the AI speaker,
the user terminal provides real-time posture data of the user periodically on a daily, weekly, or monthly basis,
the posture correction exercise recommendation device trains an AI program of an AI chatbot with answers to various questions in advance so that the user terminal receives an answer to a question from the posture correction exercise recommendation device,
the posture correction exercise recommendation device provides analysis results related to each individual's posture habit on the basis of data collection and analysis to the user terminals when the users carrying the user terminals request the analysis results, and
the posture correction exercise recommendation device recognizes a current state of the user according to a type of wearable device and recommends an exercise, which is appropriate for the user in the current state, to the user.

8. The method of claim 7, wherein the recommending of the customized posture correction exercise comprises collecting sensing data obtained by measuring posture forward or backward, left or right, and in 360° directions from a belt-shaped posture measurement device that is worn on a center of a user's chest.

9. The method of claim 7, wherein the recommending of the customized posture correction exercise comprises generating and providing images that visualize directions in which the users' bodies tilt in the 360° directions according to service requests from the user terminals.

10. The method of claim 7, wherein the recommending of the customized posture correction exercise comprises visualizing and providing daily activity data, which is generated by separating a time of maintaining correct posture from a total usage time of the wearable device for posture measurement, as a graph according to a service request from the user terminal.

11. The method of claim 7, wherein the recommending of the customized posture correction exercise comprises, when the customized posture correction exercise is recommended to the user terminal of each of the users, providing a video related to the posture correction exercise or transmitting uniform resource locator (URL) information for accessing the posture correction exercise to the user terminal.

12. The method of claim 7, wherein the recommending of the customized posture correction exercise comprises providing a notification to the user terminal of the user when the user wearing the wearable device deviates from a preset standard posture, and scoring and notifying of each user's daily posture when there is a request from the user terminal.
